(19) United States
(12) Patent Application Publication
(10) Pub. No.: US 2019/0209430 A1
(43) Pub. Date: Jul. 11, 2019

(54) INTEGRATED LAMP UNIT AND LIQUID-CIRCULATING DUAL PIPE LAMP HAVING SAME

(71) Applicant: Woo Joo Lee, Gyeonggi-do (KR)

(72) Inventor: Woo Joo Lee, Gyeonggi-do (KR)

(21) Appl. No.: 16/329,378

(22) PCT Filed: Feb. 1, 2018
(86) PCT No.: PCT/KR2018/001363

§ 371 Date: Feb. 28, 2019

(30) Foreign Application Priority Data
Feb. 22, 2017 (KR) 10-2017-0023317

(51) Int. Cl. A61H 33/06; A61N 5/06; F24H 1/10; H05B 3/00

(52) U.S. Cl. CPC A61H 33/063; A61N 5/0625; F24H 1/101; H05B 2203/032; A61H 2033/061; A61N 2005/0659; H05B 3/0033

(57) ABSTRACT

The present invention relates to an integrated liquid-circulating dual pipe lamp comprising: a lamp unit which has a plurality of coils and filaments thereinside, and which has a position-fixing ring for fixing positions of the filaments; a lamp housing which has the lamp unit thereinside, and which has a circulation hole formed thereon so that liquid circulates outside the lamp unit at a predetermined temperature; watertight members which seal both end parts of the lamp housing, and into/through which both end parts of the lamp unit are inserted and penetrate; a liquid-circulation member which is coupled to the watertight member, which supplies a predetermined amount of liquid towards the circulation hole so as to circulate the same, and which discharges the circulating liquid according to the temperature of the liquid; and a reflection member in which the lamp housing is mounted.

### TECHNICAL FIELD

The present invention relates to an integrated liquid-circulating unit and a liquid-circulating dual pipe lamp having the same. More particularly, the present invention relates to an integrated liquid lamp unit provided in a place such as sauna and the like and provides greatly facilitated maintenance and repair and management of a dual pipe lamp by allowing easy and convenient replacement of a dual pipe lamp that emits rays beneficial to human health such as near-infrared rays, and the like, and also allowing separate removal and replacement of a lamp module only, when the dual pipe lamp has deteriorating emission efficiency due to breakage or damage, and the like, and a liquid-circulating dual pipe lamp having the same.

### BACKGROUND ART

Generally, sauna is a sealed place that is maintained at high temperature with the heating pipes and electric heaters and the like provided therein, to induce perspiration of a bather, thus allowing waste to be discharged from the body, and recently, to emit infrared rays, the sauna rooms are also provided with germaniums, charcoals, and the like that emit far infrared rays into the sauna rooms.

As an example, related technology can be found in Korean Utility Model No. 20-0439309, titled “Electric heater with far infrared ray lamp for use in sauna”), which discloses a configuration that includes an insulating plate groove at a center of a front side of the electric heater, an insulating plate fit in the insulating plate groove, and a far infrared ray lamp and an electricity supply provided inside the insulating plate to seal a radiating heat inside the electric heater. However, Korean Utility Model No. 20-0439309 relates to a heater for use in sauna, which is generally aimed to increase inner temperature of a sauna room by way of heating with electric coil and blower, and thus, there is a problem that the structure cannot be installed nearby a bather due to high temperature heat generated from the heater itself, and not able to provide even distribution of radiation over the bather's body, despite the emission of infrared rays from the far infrared ray lamp. Accordingly, effects of the infrared rays are reduced.

Further, Korean Patent Registration No. 10-1558790, titled “Infrared ray lamp pipe” discloses a configuration that includes a case which is provided in a tubular form and which defines a space therein, an infrared ray emitter which is provided inside the case and which has a plurality of halogen lamps provided at intervals in a length direction, and lids fit on both sides of the case, in which the space of the case is filled with a filter liquid, and there is a filter liquid inlet formed in the lid that is fit on one side of the case, and a filter liquid outlet formed on the other side. However, the structure described above has a shortcoming in that it requires that the entire case be replaced even when only one of a plurality of emitters is damaged, which results in increased maintenance and repair costs, and thus significantly deteriorating efficiency of use.

### DISCLOSURE OF THE INVENTION

### Technical Problem

In order to solve the problems mentioned above, the present invention has been made in view of the background art described above, and it is an object of the present invention to provide an integrated lamp unit installed in a sauna or the like, which emits rays beneficial to human health such as near infrared rays, or the like, and which allows easy and convenient replacement of a dual pipe lamp, and a liquid-circulating dual pipe lamp having the same.

### Technical Solution

According to an aspect of the present invention, there is provided an integrated lamp unit, which may include: coils connected in series with a plurality of filaments; a power supply having the filaments and the coils thereinside, with both ends being electrically connected to the coils; a casing member having the filaments and the coils thereinside, with both ends being mounted with the power supply; and a position-fixing ring configured in tight contact with an inner circumference of the casing member to fix the filaments in position.

According to an embodiment, an integrated lamp unit is provided, which may have the power supply inside the casing member, and which may further include covering members that cover both end parts of the casing member, and sealing members attached to the covering members to seal a connection between the power supply and a power cable.

According to an embodiment, the integrated lamp unit is provided, which may include a connecting ring configured at a center of the position-fixing ring, with an inner circumference thereof being in contact with an outer circumference of the filaments, and a fixing cable configured to connect the connecting ring and the position-fixing ring.

According to an embodiment, the integrated lamp unit is provided, in which the filaments are mounted in the casing member at 50-100 mm intervals.

Further, according to another embodiment, the present invention may include: a lamp unit which has a plurality of coils and filaments thereinside, and which has a position-fixing ring for fixing positions of the filaments; a lamp housing which has the lamp unit thereinside, and which has a circulation hole formed thereon so that liquid circulates outside the lamp unit at a predetermined temperature; watertight members which seal both end parts of the lamp housing, and into/through which both end parts of the lamp unit are inserted and penetrate; a liquid-circulation member which is coupled to the watertight member, which supplies a predetermined amount of liquid towards the circulation hole so as to circulate the same, and which discharges the circulating liquid according to the temperature of the liquid; and a reflection member in which the lamp housing is mounted, and which enhances the amount of near-infrared irradiation emitted from the lamp unit.

Further, according to another embodiment, the lamp unit may include a casing member which has the coils and the filaments mounted therein, and which has a light-emitting groove to which the position-fixing ring is fixed, covering members which are integrally formed on both end parts of the casing member and which have a power supply electrically connected to the coils, and sealing members attached to end parts of the covering members to seal a connection between the power supply and a power cable.

Further, according to another embodiment, the lamp unit may further include a connecting ring which is provided at a center of the position-fixing ring and which is connected to the filaments, and a fixing cable for connecting the connecting ring and the position-fixing ring.

Further, according to another embodiment, the liquid circulation member may include a first circulation member which is connected to one side of a watertight member and which supplies and discharges a predetermined amount of liquid, and a second circulation member which is connected to the other side of the watertight member and which draws in and discharges liquid circulating the circulation hole according to a temperature of the liquid.

Further, according to another embodiment, the second circulation member may suck in and discharge the liquid when a temperature of the liquid exceeds 50° C.

Further, according to another embodiment, the reflection member may further include a reflection plate which enhances an amount of the near-infrared rays to a range of 3300 LUX-900 LUX, and a fixing flange which securely connects the reflection member to sauna equipment.

### Advantageous Effects

According to embodiments of the present invention, there is an effect of easy and convenient replacement of a dual pipe lamp which is installed in a sauna or the like and emits rays beneficial to human health such as near infrared rays, and the like.

Further, according to embodiments of the present invention, there is an effect in which the maintenance and repair of the dual pipe lamp is very easy, since it is possible to individually separate and replace a lamp module only, when the dual pipe lamp has a deteriorating emission efficiency due to breakage or damage occurred therein.

### BRIEF DESCRIPTION OF THE DRAWINGS

FIGS. 1 and 2 are perspective views illustrating an integrated lamp unit and a liquid-circulating dual pipe having the same according to an embodiment of the present invention;

FIGS. 3 and 4 are cross-sectional views illustrating main parts in assembled state, in an integrated lamp unit and a liquid-circulating dual pipe having the same according to an embodiment of the present invention; and

FIG. 5 is a cross-sectional view illustrating a position-fixing ring of an integrated lamp unit according to an embodiment of the present invention.

### BEST MODE FOR CARRYING OUT THE INVENTION

The present invention relates to an integrated liquid-circulating dual pipe lamp, which may include: a lamp unit which has a plurality of coils and filaments thereinside, and which has a position-fixing ring for fixing positions of the filaments; a lamp housing which has the lamp unit thereinside, and which has a circulation hole formed thereon so that liquid circulates outside the lamp unit at a predetermined temperature; watertight members which seal both end parts of the lamp housing, and into/through which both end parts of the lamp unit are inserted and penetrate; a liquid-circulation member which is coupled to the watertight member, which supplies a predetermined amount of liquid towards the circulation hole so as to circulate the same, and which discharges the circulating liquid according to the temperature of the liquid; and a reflection member in which the lamp housing is mounted, and which enhances the amount of near-infrared irradiation emitted from the lamp unit.

### MODE FOR CARRYING OUT THE INVENTION

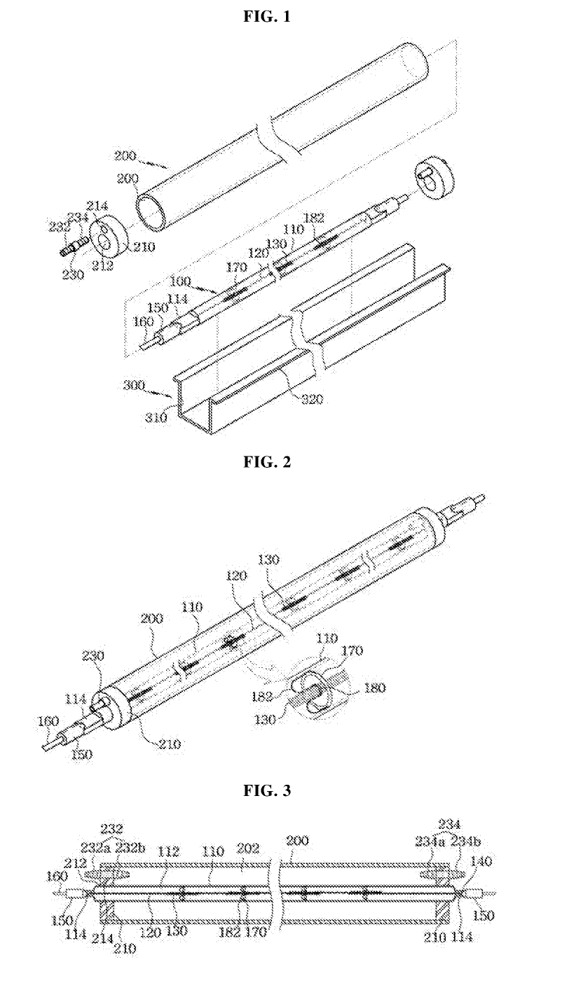

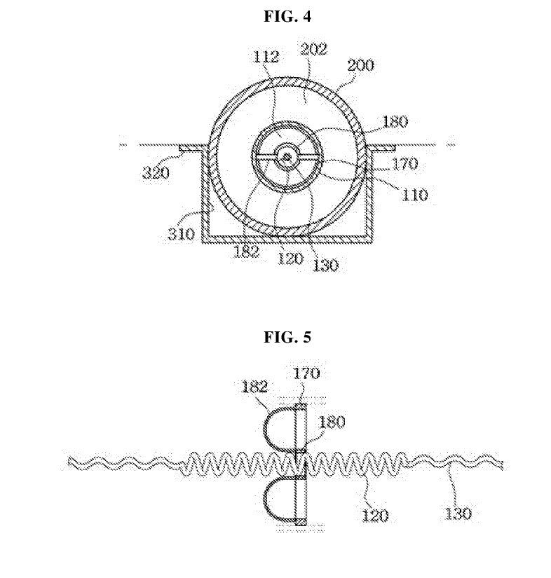

FIGS. 1 and 2 are perspective views illustrating an integrated lamp unit and a liquid-circulating dual pipe having the same according to an embodiment of the present invention, FIGS. 3 and 4 are cross-sectional views illustrating main parts in assembled state, in the integrated lamp unit and the liquid-circulating dual pipe having the same according to an embodiment of the present invention, and FIG. 5 is a cross-sectional view illustrating a position-fixing ring of the integrated lamp unit according to an embodiment of the present invention.

As illustrated, the integrated liquid-circulating dual pipe lamp according to an embodiment of the present invention includes a lamp unit 100, a lamp housing 200, and a reflection member 300.

The lamp unit 100 includes a casing member 110 having a light emitting groove 112 which is formed therein and which has coils 120 and filaments 130 mounted therein, a position-fixing ring 170 provided in the casing member 110 to prevent the filament 130 from being damaged by preventing a movement due to external impact, and the like, and a power supply 140 that supplies a predetermined power to the coils 120.

The casing member 110 includes covering members 114 on both end parts, which are electrically connected to the coils 120 and which have the power supply 140 provided thereon to supply external power.

The covering members 114 are integrally formed on both end parts of the casing member 110 and completely cover the light emitting groove 112 to prevent ingress of water that circulates outside the lamp unit 100.

Such covering members 114 are configured such that end parts of the coils 120 are inserted therein, thus electrically connecting the coils 120 and the power supply 140.

Further, a connection is formed between the power cable 160 and the power supply 140 at the end parts of the covering members 114, and there also is a sealing member 150 provided to seal the connection between the power cable 160 and the power supply 140.

The sealing member 150 is configured such that one end part is attached to an end part of the covering member 114, while the other end part is inserted into the power cable 160 to seal the connection between the power supply 140 and the power cable 160 provided in the covering member 114.

There may be a plurality of position-fixing rings 170 provided at predetermined intervals inside the casing member 110, and further, the position-fixing rings 170 may be provided in the same number as the filaments 130 to fix the positions of the filaments 130 connected to the coils 120.

Such a position-fixing ring 170 is configured such that the position-fixing ring 170 is in close contact with an inner circumference of the casing member 110, and the filaments 130 are penetrated through a center thereof.

Further, the position-fixing ring 170 additionally includes, at a center thereof, a connecting ring 180 in contact with an outer circumference of the filaments 130, and the connecting ring 180 includes a fixing cable 182 to connect to the position-fixing ring 170.

The fixing cable 182 is formed of a metal material and connects the connecting ring 180 and the position-fixing ring 170 to maintain the connecting ring 180 in the state of being fixed at the center of the position-fixing ring 170.

As a result, because the positions of the filaments 130 connected to the connecting ring 180 can be maintained to be fixed in the center of the position-fixing ring 170, damages of the filaments 130 can be prevented even when external impact occurs.

Here, there may be a plurality of filaments 130 provided at predetermined intervals and connected in series to the coils 120 such that, when electric currents are applied, the filaments 130 emit infrared rays, which alleviate acute or chronic pains generated at muscle or nerve tissues of the spines of patients, and provide thermal stimulus on the sites where the pains are generated, to thus improve blood circulation or resolve stiffness in the muscle, or the like.

At this time, the filaments 130 may be mounted in the casing member 110 at 50-100 mm intervals to achieve the even distribution of the infrared irradiation over respective body parts of an adult user of the sauna, or the like.

The lamp housing 200 is provided inside the lamp unit 100 and includes a circulation hole 202 formed thereon, through which liquid circulates outside the lamp unit 100 at a predetermined temperature. Accordingly, when the infrared rays are emitted from the lamp unit 100, the circulation hole 202 plays a role of allowing a predetermined amount of liquid to flow, thus allowing only the near-infrared rays to be delivered to the user.

Such a lamp housing 200 is formed in a hollow tube shape with both open end parts, and formed from a transparent material such as acryl to allow the infrared rays emitted from the lamp unit 100 to be passed therethrough.

Here, watertight members 210 are configured on the both open end parts of the lamp housing 200 to fix the position of the lamp unit 100 and to prevent leakage of the liquid such as water that circulates through the circulation hole 202.

The watertight member 210 is formed from silicone, and the like and completely seals both end parts of the lamp housing 200, and includes a lamp coupling hole 212 formed at a center, through which the covering member 114 of the lamp unit 100 is passed to be exposed outside, thus allowing a supply of external power.

The lamp coupling hole 212 may be formed in a same diameter as a diameter of the casing member 110 of the lamp unit 100, or may be formed in a diameter smaller than that of the casing member 110, and configured such that, when end parts of the casing member 110 are inserted and passed through, a tight coupling is formed with the watertight member 210 to prevent the liquid circulating through the circulation hole 202 from leaking through seams between the lamp coupling hole 212 and the casing member 110.

Further, the watertight member 210 includes a passing hole 214 through which a liquid circulation member 230 to be described below is inserted and passed, and the passing hole 214 is also preferably configured to form a tight coupling with the liquid circulation member 230.

The liquid circulation member 230 is an element provided to supply a predetermined amount of water toward a circulation hole 303 formed on the lamp housing 200 and discharge it, thus providing a circulation by the flow of the liquid.

Such a liquid circulation member 230 may include first and second circulation members 232, 234 provided on one and the other sides of the watertight member 210, respectively.

Although not illustrated, the first circulation member 232 includes a supply part 232a connected to a liquid storage tank where the liquid such as water is stored to be supplied with a predetermined amount of water, and a discharge part 232b that discharges the water supplied from the supply part 232a toward the circulation hole 202.

Further, the second circulation member 234 is provided to maintain the liquid circulating within the circulation hole 202 at a constant temperature as the liquid circulating through the circulation hole 202 is heated to a predetermined temperature or higher by the infrared rays emitted from the lamp unit 100, and includes a suction part 234a that sucks in the liquid, and a discharge part 234b that discharges the sucked liquid.

Here, the second circulation member 234 may be configured to be connected to the element such as a suction pump, a circulation pump, or the like such that the liquid can be sucked and discharged, and the discharge part 234b may be configured such that the liquid being discharged is discharged back to the liquid storage tank and cooled down to a predetermined temperature, and then re-supplied into the circulation hole 202 by the first circulation member 232.

At this time, the second circulation member 234 may additionally include a temperature sensor to ensure that the liquid circulating within the circulation hole 202 is circulated only when at a temperature range from minimum 30° C. to maximum 50° C. and that the liquid is discharged through the second circulation member 234 only when the temperature exceeds 50° C.

The reflection member 300 is an element configured such that, as a lower portion of the lamp housing 200 is inserted and coupled with the reflection member 300, the amount of the near-infrared rays emitted from the lamp unit 100 is maximized, and also the lamp housing 200 with the lamp unit 100 mounted therein is enabled to be installed in the equipment such as sauna equipment and the like.

Such a reflection member 300 is provided with a reflection plate 310 on an inner surface to enhance the amount of the near-infrared rays emitted from the lamp unit 100 so that the near-infrared rays are emitted at a range of 3300 LUX-4900 LUX, and additionally includes fixing flanges 320 formed on both upper ends to securely connect the lamp unit 100 and the lamp housing 200 according to the present invention to the sauna equipment, and the like.

According to the present invention configured as described above, when damage occurs at the filaments 130 or the coils 120 provided inside the lamp unit 100, only the lamp unit 100 may be individually separated from the lamp housing 200 and replaced. Accordingly, the present invention will overcome the problem of the related art that requires that the lamp assembly with all the infrared-emitting components connected thereto be replaced.

Accordingly, the present invention can be provided in a place such as sauna and the like, and provides greatly facilitated maintenance and repair and management of a dual pipe lamp by allowing easy and convenient replacement of the dual pipe lamp that emits rays beneficial to human health such as near-infrared rays, and the like, and also allows separate removal and replacement of a lamp module only, when the dual pipe lamp has deteriorating emission efficiency due to breakage or damage, and the like.

It is to be understood that the terms “comprise”, “include”, or “have” as used herein may mean that the component can be inherently included unless specifically stated to the contrary, and therefore, should be construed as further including other elements rather than exclude other components, all terms including technical or scientific terms have the same meaning as commonly understood by one of ordinary skill in the art to which this invention belongs, unless otherwise defined.

In addition, the present disclosure has been described in detail. However, it should be understood that the detailed description and specific examples, while indicating preferred embodiments of the disclosure, are given by way of illustration only, since various changes and modifications within the scope of the disclosure will become apparent to those skilled in the art from this detailed description. Accordingly, the embodiments disclosed in the present disclosure are provided to explain, not to limit a technical art of the present disclosure, and thus, the scope of the present disclosure is not limited. The scope of the present disclosure should be construed by following claims, and every technical art within a same scope of the following claims should be construed to be included in a scope of a right of the present disclosure.

### INDUSTRIAL APPLICABILITY

The present invention can be provided in a place such as sauna and the like, and provides greatly facilitated maintenance and repair and management of a dual pipe lamp by allowing easy and convenient replacement of the dual pipe lamp that emits rays beneficial to human health such as near-infrared rays, and the like.

DESCRIPTION OF REFERENCE NUMERALS

| | |
|---|---|
| 100: Lamp unit | 110: Casing member |
| 120: Coil | 130: Filament |
| 140: Power supply | 150: Sealing member |
| 160: Power cable | 170: Position-fixing ring |
| 180: Connecting ring | 200: Lamp housing |
| 210: Watertight member | 230: Liquid circulation member |
| 300: Reflection member | 310: Reflection plate |
| 320: Fixing flange | |

1. An integrated lamp unit, comprising:
coils connected in series with a plurality of filaments;
a power supply having the filaments and the coils thereinside, with both ends being electrically connected to the coils;
a casing member having the filaments and the coils thereinside, with both ends being mounted with the power supply; and
a position-fixing ring configured in tight contact with an inner circumference of the casing member to fix the filaments in position.

2. The integrated lamp unit of claim 1,
wherein the power supply is provided inside the casing member,
wherein the integrated lamp unit further comprising:
covering members that cover both end parts of the casing member; and
sealing members attached to the covering members to seal a connection between the power supply and a power cable.

3. The integrated lamp unit of claim 1, further comprising:
a connecting ring configured at a center of the position-fixing ring, with an inner circumference thereof being in contact with an outer circumference of the filaments; and
a fixing cable configured to connect the connecting ring and the position-fixing ring.

4. The integrated lamp unit of claim 1, wherein the filaments are mounted in the casing member at 50-100 mm intervals.

5. An integrated liquid-circulating dual pipe lamp, comprising:
a lamp unit which has a plurality of coils and filaments thereinside, and which has a position-fixing ring for fixing positions of the filaments;
a lamp housing which has the lamp unit provided thereinside and connected thereto, and which has a circulation hole formed thereon so that liquid circulates outside the lamp unit at a predetermined temperature;
a liquid-circulation member which is configured on both end parts of the lamp housing to supply a predetermined amount of liquid and circulate the same, and discharge the circulating liquid according to the temperature of the liquid; and
a reflection member in which the lamp housing is mounted, and which enhances the amount of near-infrared irradiation emitted from the lamp unit.

6. The integrated liquid-circulating dual pipe lamp of claim 5, further comprising:
watertight members for sealing both end parts of the lamp housing, wherein the watertight members including lamp coupling holes into/through which both end part of the lamp unit are inserted and penetrated; and
a passing hole to which the liquid circulation member is connected.

7. The integrated liquid-circulating dual pipe lamp of claim 5, further comprising:
a casing member which has the coils and the filaments mounted therein, and which has a light-emitting groove to which the position-fixing ring is fixed;
covering members which are integrally formed on both end parts of the casing member and which have a power supply electrically connected to the coils; and
sealing members attached to end parts of the covering members to seal a connection between the power supply and a power cable.

8. The integrated liquid-circulating dual pipe lamp of claim 7, wherein the lamp unit further comprising:
a connecting ring which is provided at a center of the position-fixing ring and which is connected to the filaments; and
a fixing cable for connecting the connecting ring and the position-fixing ring.

9. The integrated liquid-circulating dual pipe lamp of claim 5, wherein the liquid circulation member comprising:
a first circulation member which is connected to one side of a watertight member and which supplies and discharges a predetermined amount of liquid; and
a second circulation member which is connected to the other side of the watertight member and which sucks in and discharges a liquid circulating the circulation hole according to a temperature of the liquid.

10. The integrated liquid-circulating dual pipe lamp of claim 9, wherein the second circulation member sucks in and discharges the liquid when a temperature of the liquid exceeds 50° C.

11. The integrated liquid-circulating dual pipe lamp of claim 5, wherein the reflection member further comprising:
a reflection plate which enhances an amount of the near-infrared rays to a range of 3300 LUX-900 LUX; and
a fixing flange which securely connects the reflection member to sauna equipment.

12. An integrated lamp unit, comprising:
coils connected in series with a plurality of filaments;
a power supply having the filaments and the coils thereinside;
a casing member having the filaments and the coils thereinside; and
a position-fixing ring configured in tight contact with an inner circumference of the casing member.

13. The integrated lamp unit of claim 12, wherein the power supply is provided inside the casing member.

14. The integrated lamp unit of claim 12, further comprising:
covering members that cover both end parts of the casing member; and
sealing members attached to the covering members to seal a connection between the power supply and a power cable.

15. The integrated lamp unit of claim 12, further comprising:
a connecting ring configured at a center of the position-fixing ring, with an inner circumference thereof being in contact with an outer circumference of the filaments; and
a fixing cable configured to connect the connecting ring and the position-fixing ring.

16. The integrated lamp unit of claim 12, wherein the filaments are mounted in the casing member at 50-100 mm intervals.
